(19) United States
(12) Patent Application Publication
(10) Pub. No.: US 2025/0261871 A1
(43) Pub. Date: Aug. 21, 2025

(54) ESOPHAGEAL PRESSURE MEASUREMENT CATHETER

(71) Applicant: PULMOTECH B.V., Groningen (NL)

(72) Inventors: Rutger Christiaan FLINK, Groningen (NL); Laurens Bastiaan Josef HOGENBIRK, Groningen (NL); Teunis Hendrik SCHAART, Groningen (NL)

(73) Assignee: PULMOTECH B.V., Groningen (NL)

(21) Appl. No.: 19/063,801

(22) Filed: Feb. 26, 2025

Related U.S. Application Data
(63) Continuation of application No. 17/434,452, filed on Aug. 27, 2021, now abandoned.

(30) Foreign Application Priority Data
Feb. 28, 2019 (NL) 2022650

(51) Int. Cl. A61B 5/03; A61B 5/00

(52) U.S. Cl. CPC A61B 5/037; A61B 5/6853; A61B 2562/0247

(57) ABSTRACT

An esophageal pressure measurement catheter includes a tube having a proximal end and a distal end. The distal end of the tube is arranged to be inserted into the esophagus of a patient. The tube is provided with at least one pressure sensor for measuring a pressure in the esophagus region of a patient. The tube is provided with at least one inflatable balloon adjacent to the at least one pressure sensor. A distance between an edge of the balloon and the centre of the pressure sensor is less than 20 mm.

### CROSS-REFERENCE TO RELATED APPLICATIONS

This is a continuation of co-pending U.S. patent application Ser. No. 17/434,452, filed Aug. 27, 2021, with is a national stage application filed under 35 U.S.C. § 371 of international application PCT/EP2020/054922, filed Feb. 25, 2020, which claims priority to Netherlands Patent Application No. NL 2022650, filed Feb. 28, 2019, the entirety of which applications are hereby incorporated by reference herein.

### FIELD OF THE DISCLOSURE

The invention relates to a catheter comprising a tube having a proximal end and a distal end, wherein the tube is provided with at least one pressure sensor for measuring a pressure, and wherein the tube is provided with at least one inflatable balloon adjacent to the at least one pressure sensor.

### BACKGROUND OF THE DISCLOSURE

Such a catheter is disclosed in US 2017/136220 A1, which relates generally to catheter devices for the site-specific delivery of agents to biological spaces such as blood vessels for treatment of said blood vessels and/or other organ systems. In other embodiments other target spaces that may be accessed by the distal end of the catheter include the gastroesophageal tract. The catheter is designed with at least two occlusion balloons, one proximal and one distal, which are large enough so as to produce a closed off agent delivery segment. The catheter optionally comprise a pressure sensing means, whereby the pressure of the fluid environment in the closed off agent delivery segment can be measured.

The current invention relates to different type of catheters, namely an esophageal pressure measurement catheter which is specifically designed for measuring pressures in a patient's esophagus. A problem when measuring pressures in a patient's esophagus is that any interaction between the esophagus wall and the pressure sensor, such as the wall touching the pressure sensor, may influence the measurement in a negative manner. The invention aims at a more reliable esophageal pressure measurement catheter.

To that end, the tube comprises a feeding lumen for transporting liquid food, medication and/or gastric acid to and/or from the stomach of a patient, the balloon is inflatable to a diameter of between 7 mm and 18 mm, and a distance between an edge of the balloon and the centre of the pressure sensor is less than 20 mm. The balloon, when inflated, acts as a spacer to keep the wall of the esophagus away from the pressure sensor, such that the measurement is more accurate.

Said at least one pressure sensor preferably is a solid state pressure sensor. The tube preferably comprises a pressure sensor lumen for accommodating a connection for the at least one pressure sensor. The tube preferably comprises a balloon inflation lumen for transporting a fluid, such as air, to the at least one inflatable balloon.

The balloon, when inflated under normal, nominally designed operating conditions, preferably has a diameter of between 7 mm and 18 mm, more preferably between 10 mm and 15 mm. The distance between an edge of the balloon and the centre of the pressure sensor is preferably less than 10 mm, more preferably 7 mm, even more preferably less than 5 mm, most preferably less than 3 mm. Preferably the balloon has a spherical shape, or the balloon has a cylindrical shape with a substantially rectangular longitudinal cross section. Preferably the balloon is located at the side of the pressure sensor that is nearer to the proximal end than to the distal end of the tube.

The balloon is preferably made of urethane, PET and/or nylon. The wall thickness of the balloon is preferably between 0.01 mm and 0.06 mm. The balloon may have a leg on either side for fixating the balloon on the tube, and said leg is less preferably than 7 mm, more preferably less than 5 mm, most preferably less than 3 mm.

The length of the tube is preferably at least 80 cm, preferably at least 95 cm. The diameter of the tube is preferably between 2 mm and 6 mm. The tube is preferably provided with two of said inflatable balloons adjacent and at each side of the at least one pressure sensor, wherein a distance between an edge of each of the balloons and the centre of the pressure sensor is less than 20 mm, preferably less than 10 mm, more preferably less than 7 mm, even more preferably less than 5 mm, most preferably less than 3 mm. Said tube is preferably provided with a plurality of said pressure sensors distributed along a length of the tube, each sensor being provided with at least one inflatable balloon adjacent the at least one pressure sensor, wherein a distance between an edge of the balloon and the centre of the pressure sensor is less than 20 mm, preferably less than 10 mm, more preferably less than 7 mm, even more preferably less than 5 mm, most preferably less than 3 mm. At least one of said pressure sensors is preferably provided near the distal end of the tube, for instance in the last 25%, 10% or 5% of the length of the tube.

### BRIEF DESCRIPTION OF THE DRAWINGS

The invention will now be exemplified by means of preferred embodiments, with reference to the drawings, in which:

FIG. 1 is a side view of an esophageal pressure measurement catheter in accordance with the invention;

FIG. 2 is a top view of the esophageal pressure measurement catheter of FIG. 1;

FIG. 3 is a cross section of the esophageal pressure measurement catheter of FIG. 1;

FIG. 4 is a side view of a balloon for use with the esophageal pressure measurement catheter in accordance with the invention; and

FIG. 5 is a side view of another embodiment of a balloon for use with the esophageal pressure measurement catheter in accordance with the invention.

### DETAILED DESCRIPTION

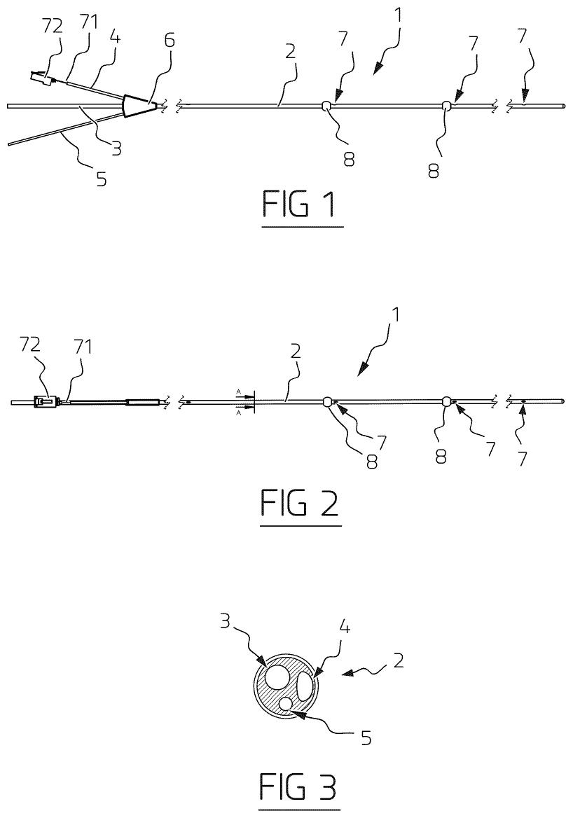

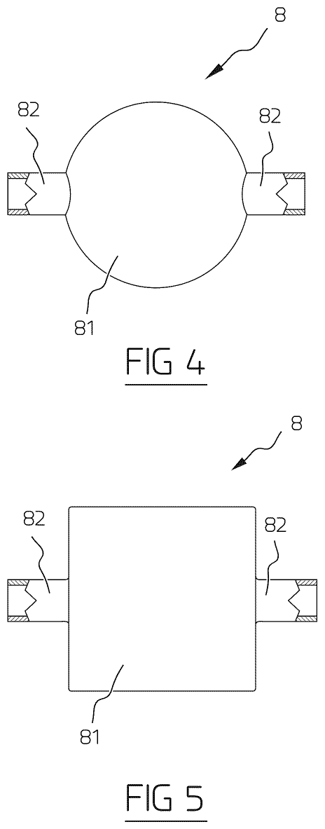

According to FIGS. 1 to 3 the esophageal pressure measurement catheter 1 comprises a flexible tube 2. The tube is typically 95-125 cm long and approximately 4 mm in 5 diameter. The tube is provided with an enteral feeding lumen 3, a sensor lumen 4 and a balloon inflation lumen 5, which exit the tube 2 through a trifurcation piece 6.

The enteral feeding lumen 3 is used to feed a patient with liquid food, administer medication and/or suction aspirate during the pressure measurement period, which may last for more than several days.

The pressure is measured by a plurality of solid state pressure sensors 7, which are distributed along the length of the tube 2 and which are in open communication with the esophagus of the patient though corresponding holes in the wall of the tube 2. The sensors 7 are connected by connection members 71 in the sensor lumen 4 to a sensor connector 72, for instance an RJ45 connector, at the proximal outer end of the sensor lumen 4.

Adjacent each pressure sensor 7 an inflatable medical balloon 8 is provided on the tube 2. The balloons 8 may for instance be made of urethane. The distance between the edge of the inflated balloon 8 and the centre of the pressure sensor is for instance approximately 4 mm, while the diameter of the inflated balloon is for instance approximately 12 to 15 mm. In that manner the balloon acts as a spacer, which prevents that the wall of the esophagus can touch or come near the pressure sensor 7.

In another preferred embodiment, a balloon 8 is provided at each side of each pressure sensor 7 at about 3 mm distance, whereby the spacer function that prevents that the wall of the esophagus can touch or come near the pressure sensor 7 is improved compared to the embodiment with one balloon 8 for each pressure sensor 7.

FIG. 4 shows an example of a balloon 8, which comprises a spherical inflatable part 81 and two leg parts 82 at each end for mounting the balloon 8 on the tube 2. The leg parts are about 3 mm long in the axial direction.

FIG. 5 shows another example of a balloon 8, which comprises a cylindrical inflatable part 81 and two leg parts 82 at each end for mounting the balloon 8 on the tube 2. The leg parts are about 3 mm long in the axial direction. The inflatable part 81 has a substantially rectangular longitudinal cross section, whereby the spacer function that prevents that the wall of the esophagus can touch or come near the pressure sensor 7 is improved compared to the embodiment of FIG. 4.

The invention has thus been described by means of preferred embodiments. It is to be understood, however, that this disclosure is merely illustrative. Various details of the structure and function were presented, but changes made therein, to the full extent extended by the general meaning of the terms in which the appended claims are expressed, are understood to be within the principle of the present invention. The description and drawings shall be used to interpret the claims. The claims should not be interpreted as meaning that the extent of the protection sought is to be understood as that defined by the strict, literal meaning of the wording used in the claims, the description and drawings being employed only for the purpose of resolving an ambiguity found in the claims. For the purpose of determining the extent of protection sought by the claims, due account shall be taken of any element which is equivalent to an element specified therein. An element is to be considered equivalent to an element specified in the claims at least if said element performs substantially the same function in substantially the same way to yield substantially the same result as the element specified in the claims.

1-14. (canceled)

15. An esophageal pressure measurement catheter comprising:
a tube having a proximal end and a distal end,
wherein the distal end of the tube is arranged to be inserted into an internal region of the esophagus of a patient,
wherein the tube is provided with at least one pressure transducer for measuring a pressure in the internal region of the esophagus of the patient, wherein the at least one pressure transducer is provided in a wall of the tube so as to be in direct communication with said internal region of the esophagus of the patient,
wherein the tube is provided with at least one inflatable balloon adjacent to the at least one pressure transducer,
wherein the catheter is an esophageal pressure measurement catheter,
wherein the tube comprises a feeding lumen for transporting liquid food, medication and/or gastric acid to and/or from the stomach of a patient,
wherein the balloon is inflatable to a diameter of between 7 mm and 18 mm, and
wherein the balloon is arranged at a distance from the pressure transducer along the length of the tube, such that a distance between an edge of the balloon and the center of the pressure transducer is less than 20 mm, the balloon, when inflated, acting as a spacer to keep the wall of the esophagus away from the pressure transducer.

16. The esophageal pressure measurement catheter in accordance with claim 15, wherein the tube comprises a pressure transducer lumen for accommodating a connection for the at least one pressure transducer.

17. The esophageal pressure measurement catheter in accordance with claim 15, wherein the tube comprises a balloon inflation lumen for transporting a fluid to the at least one inflatable balloon.

18. The esophageal pressure measurement catheter in accordance with claim 15, wherein the balloon is inflatable to a diameter of between 10 mm and 15 mm.

19. The esophageal pressure measurement catheter in accordance with claim 15, wherein the balloon has a spherical shape, or the balloon has a cylindrical shape with a substantially rectangular longitudinal cross section.

20. The esophageal pressure measurement catheter in accordance with claim 15, wherein the length of the tube is at least 80 cm.

21. The esophageal pressure measurement catheter in accordance with claim 15, wherein the diameter of the tube is between 2 mm and 6 mm.

22. The esophageal pressure measurement catheter in accordance with claim 15, wherein said at least one pressure transducer is a solid state pressure transducer.

23. The esophageal pressure measurement catheter in accordance with claim 15, wherein the balloon is made of urethane, polyethylene terephthalate and/or nylon.

24. The esophageal pressure measurement catheter in accordance with claim 15, wherein the wall thickness of the balloon is between 0.01 mm and 0.06 mm.

25. The esophageal pressure measurement catheter in accordance with claim 15, wherein the balloon has a leg on either side of said balloon for fixating the balloon on the tube, and wherein the length of each leg is less than 7 mm in axial direction.

26. The esophageal pressure measurement catheter in accordance with claim 15, wherein the tube is provided with two of said inflatable balloons adjacent to and at each side of the at least one pressure transducer, wherein a distance between an edge of each of the balloons and the center of the pressure transducer is less than 20 mm.

27. The esophageal pressure measurement catheter in accordance with claim 15, wherein said tube is provided with a plurality of said pressure transducers distributed along a length of the tube, each transducer being provided with at least one inflatable balloon adjacent to the at least one pressure transducer, and wherein a distance between an edge of the balloon and the center of the pressure transducer is less than 20 mm.

28. The esophageal pressure measurement catheter in accordance with claim 15, wherein the distance between an edge of the balloon and the center of the pressure sensor is less 7 mm.

29. The esophageal pressure measurement catheter in accordance with claim 20, wherein the length of the tube is at least 95 cm.

30. The esophageal pressure measurement catheter in accordance with claim 25, wherein the length of said leg is less than 5 mm.

31. The esophageal pressure measurement catheter in accordance with claim 26, wherein the distance between an edge of each of the balloons and the center of the pressure sensor is less than 10 mm.

32. The esophageal pressure measurement catheter in accordance with claim 27, wherein the distance between an edge of the balloon and the center of the pressure sensor is less than 10 mm.

33. A catheter comprising:
a tube having a proximal end and a distal end,
wherein the distal end of the tube is arranged to be inserted into an internal region of the esophagus of a patient,
wherein the tube is provided with at least one pressure transducer for measuring a pressure in the internal region of the esophagus of the patient, wherein the at least one pressure transducer is provided in a wall of the tube so as to be in direct communication with said internal region of the esophagus of the patient,
wherein the tube is provided with at least one inflatable balloon adjacent to the at least one pressure transducer,
wherein the catheter is an esophageal pressure measurement catheter,
wherein the tube comprises a feeding lumen for transporting liquid food, medication and/or gastric acid to and/or from the stomach of a patient,
wherein the balloon is inflatable to a diameter of between 7 mm and 18 mm,
wherein a distance between an edge of the balloon and the center of the pressure transducer is less than 20 mm, the balloon, when inflated, acting as a spacer to keep the wall of the esophagus away from the pressure transducer,
wherein the pressure sensor is connected by a connection member and the tube comprises a pressure sensor lumen for accommodating the connection member, and
wherein the tube further comprises a balloon inflation lumen for transporting a fluid to the at least one inflatable balloon, said balloon inflation lumen being different from the pressure transducer lumen.

34. A catheter comprising:
a tube having a proximal end and a distal end,
wherein the distal end of the tube is arranged to be inserted into an internal region of the esophagus of a patient,
wherein the tube is provided with at least one pressure transducer for measuring a pressure in the internal region of the esophagus of the patient, wherein the at least one pressure transducer is provided in a wall of the tube so as to be in direct communication with said internal region of the esophagus of the patient,
wherein the tube is provided with a spacer to keep the wall of the esophagus away from the pressure transducer arranged adjacent to the at least one pressure transducer,
wherein the catheter is an esophageal pressure measurement catheter, and
wherein the tube comprises a feeding lumen for transporting liquid food, medication and/or gastric acid to and/or from the stomach of a patient.
